SEQUENCE LISTING (6 sequences)

SEQ ID NO: 1 - DNA, 30 bp, Artificial Sequence
  It is synthesized.
actgataacgtaggaacctggacccttgat

SEQ ID NO: 2 - DNA, 47 bp, Artificial Sequence
  It is synthesized.
actgataacgtaggaacctggacccttgatcgggtgtgaccaacatc

SEQ ID NO: 3 - DNA, 92 bp, Artificial Sequence
  It is synthesized.
atggtgcacctgactcctgaggagaagtctgccgttactgccctgtggggcaaggtgaacgtggattaagttggtggtgaggccctgggcag

SEQ ID NO: 4 - DNA, 92 bp, Artificial Sequence
  It is synthesized.
atggtgcacttgacttctgaggagaagaactgcatcactaccatctggtctaaggtgcaggttgaccagactggtggtgaggcccttggcag

SEQ ID NO: 5 - DNA, 92 bp, Artificial Sequence
  It is synthesized.
atggtgcacctaactgatgctgagaaggctactgttagtggcctgtggggaaaggtgaaccctgataatgttggcgctgaggccctgggcag

SEQ ID NO: 6 - DNA, 105 bp, Artificial Sequence
  It is synthesized.
atggtgcacctgactcctgaggagaagtctgccgttactgccctgtggggcaaggtgaacgtggatgaagttggtggtgaggccctgggcaggttggtatcaagg

(19) United States
(12) Patent Application Publication
(10) Pub. No.: US 2022/0101949 A1
(43) Pub. Date: Mar. 31, 2022

(54) Similarity analysis method of negative sequential patterns based on biological sequences and its implementation system and medium

(71) Applicant: QILU UNIVERSITY OF TECHNOLOGY, Jinan (CN)

(72) Inventors: Xiangjun DONG, Jinan (CN); Yue LU, Jinan (CN)

(21) Appl. No.: 17/446,176

(22) Filed: Aug. 27, 2021

Related U.S. Application Data
(63) Continuation of application No. PCT/CN2020/128253, filed on Nov. 12, 2020.

(30) Foreign Application Priority Data
Sep. 25, 2020 (CN) 202011022788.8

(51) Int. Cl. G16B 30/10; G16B 40/30; G16B 45/00; G16B 50/00

(52) U.S. Cl. CPC G16B 30/10; G16B 50/00; G16B 45/00; G16B 40/30

(57) ABSTRACT

A similarity analysis method of negative sequential patterns based on biological sequences and its implementation system and medium comprises: (1) Data preprocessing: represent the letters in the DNA sequence with numbers; divide the sequence represented by numbers into several blocks as datasets for frequent pattern mining; (2) Frequent pattern mining: utilize the f-NSP algorithm to mine the data sets; (3) Represent the maximum frequent positive and negative sequential patterns graphically; convert the maximum frequent positive and negative sequential patterns into number sequences; (4) Similarity analysis of DNA sequence: calculate the similarity of different DNA sequences; select the DNA sequence corresponding to the minimum similarity as the sequence to be studied.

### TECHNICAL FIELD

This invention is related to a similarity analysis method of negative sequential patterns based on biological sequences and its implementation system and medium and belongs to the technical field of actionable high utility negative sequential rules.

### BACKGROUND ART

In recent years, we have obtained massive amounts of biological sequence data. With the development of the DNA and protein sequencing techniques, there is an increasing demand for data analysis tools that interpret all kinds of information contained in the biological sequence data, especially the genetic and regulatory information in DNA sequences, and the relationships between protein sequence structures and functions; and the similarity analysis of sequences has been widely used. Whenever we obtain a new DNA sequence, we always want to prove its similarity with some known sequences by similarity analysis. If it is homologous to a known sequence, we will save great time and efforts in re-determining the functions of the new sequence. This is particularly important as the number of biological sequences is huge. In the analysis of biological sequences, sequential pattern mining helps to identify concurrent biological sequences and discover relationships in the DNA or protein sequences. Therefore, studying the missing base-pair sequences is of greater significance than simply mining frequent sequential patterns. In bioinformatics researches, the similarity analysis of biological sequences is by no means a simple or mechanical comparison, but is definitely diversified, and it also needs many mathematical and statistical methods to assist in the analysis and evaluation. Sequence alignment is the most common and classic research method in analyzing sequence similarity. It is the basis of gene recognition, molecular evolution, and life origin researches to analyze the similarity of sequences from the biological sequence level and infer their structural, functional and evolutionary connections; however, there are two problems in the sequence alignment that directly affect the similarity score: substitution matrix and gap penalty. A rough alignment method only describes the relationship between two bases as the same or different. The similarity analysis of biological sequences is used to extract information stored in protein sequences, and many mathematical solutions have been put forward for this purpose. The graphical representation of a biological sequence can identify the information content of any sequence to help biologists choose another complex theoretical or experimental method. The graphical representation not only provides a visual qualitative inspection, but also provides a mathematical description through a matrix and other objects. Most mathematical solutions are based on 2-D and 3-D representations.

As for sequential pattern mining, the Positive Sequential Pattern (PSP) mining only consider the events (behaviors) that have occurred, while, distinguished from the thinking of this traditional sequential pattern mining, the Negative Sequential Pattern (NSP) mining also considers events (behaviors) that did not occur, i.e. items that do not exist in the sequences, for example, the different degrees of influence exerted by various existing situations on campus on students' study and life; the insured person who is suspected of medical fraud by eliminating the adverse records of drug purchasing; and the missing gene segments may trigger underlying diseases, etc., thus providing more comprehensive decision-making information for us. Such items are easy to be ignored by humans; therefore, they are attracting more and more attention from data mining workers. In particular, in the biological sequence analysis, sequential pattern mining helps to identify the concurrent biological sequences and discover relationships in the DNA or protein sequences. Therefore, studying the missing base-pair sequences is of greater significance than simply mining frequent sequential patterns. There are some important problems in the biological data analysis or biological data mining, such as discovering concurrent biological sequences, effective classification of biological sequences, and clustering analysis of biological sequences. The sequential pattern mining algorithm helps to identify concurrent biological sequences and discover relationships in the DNA or protein sequences. The biological sequence data often contains a wealth of valuable biological information; for example, the frequently occurring gene and protein fragments in the biological sequences often contain much unknown information, and it is of great significance to mine such information; the attack by some bacteria on the human body is affected by some fragments in their genes; and the extreme expansion of some tandem repeat sequences in variable number may lead to related neurological diseases. Additionally, the discovery of the frequent patterns in DNA sequences is an effective method to explain the biological inheritance characters, and these frequent patterns are often possible trends of implied data in the biological sequences and markers associated with certain events. Therefore, the mining of frequent patterns in the biological sequences of proteins or DNAs is of great value.

The existing similarity analysis methods mainly apply to the PSP, and they still lack a uniform similarity measurement method for the NSP we have mined earlier. Moreover, the sequence alignment has some shortcomings, which leads to an attempt to find other ways to compare the similarities of DNA sequences. We know that the existence of NSP is inevitable in the biological data and even crucial for some disease-causing genes, which forces us to find a way to perform similarity analysis on the DNA of sequences with missing bases.

### DESCRIPTION OF THE INVENTION

In view of the shortcomings of the existing technologies, the invention has presented a similarity analysis method of negative sequential patterns based on biological sequences;

The invention has also presented an implementation system for the above similarity analysis method.

To effectively analyze the similarity of DNA sequences, the following key issues should be addressed: (1) How to represent the main sequences of DNA as number sequences effectively; (2) How to obtain and select appropriate descriptors that can be regarded as characteristics of DNA sequences and represent the sequences according to the number sequences; (3) How to effectively process the DNA sequences of different lengths and keep them consistent; (4) How to perform effective similarity analysis on negative sequences.

### Term Interpretation

1. DNA sequence, also referred to as gene sequence, is the primary structure of a real or hypothetical DNA molecule that carries genetic information, which is represented by a string of letters.
2. f-NSP algorithm: f-NSP uses bitmaps to store PSP data and calculates the NSC support through bit manipulations. It creates a bitmap for a PSP with a size greater than 1. If a positive sequence is included in the ith data sequence, we set the ith position of the bitmap of the positive sequence to 1; otherwise, we set it as 0. The length of each bitmap is equal to the number of sequences contained in the data sequence. By using a new bitmap storage structure, we can replace the original union operations with bitwise OR operations. The length of each bitmap equals the number of sequences in the database. Assuming that s is a positive sequence and its bitmap is represented by B(s) and the number of “1” in the obtained bitmap is represented by N(B(s)), then for a given m-size and n-neg-size negative sequence ns, its support is:

sup(ns)=sup(MPS(ns))−N(ORᵢ₌₁ⁿ{B(p(1-negMSᵢ))}) (1)

If ns contains only one negative element, then the support of sequence ns is:

sup(ns)=sup(MPS(ns))−sup(p(ns)) (2)

Particularly, for the negative sequence <¬G>,

sup(<¬G>)=|D|−sup(<G>) (3)

that contains a single element only

The f-NSP algorithm comprises the following steps. 1. Find all PSP algorithms from the sequence database based on the GSP algorithm. All the PSPs and their bitmaps will be stored in a hash table named PSPHash; 2. Use the NSC (Negative Sequence Candidate) generation method to generate NSCs for each PSP; 3. Calculate the support of the 1-neg-size nsc with formulas (2) and (3). Then, the support of other NSCs can be easily calculated by the formula (1). To be specific, we obtain the bitmap of each 1-neg-MS' from the 1-negMSS_{nsc} first; secondly, we use OR operations to obtain the union set of the bitmaps; then, we calculate the support of nsc according to the formula (1); finally, we determine whether an NSC is an NSP by comparing its support with the min_sup. 4. Return the results and end the entire algorithm.

3. GSP algorithm: GSP algorithm is a mining algorithm based on breadth-first search strategy which obtains the frequent item sets contained in the database by scanning the database one time, then generates the candidate sequences with increasing length through the corresponding connection and pruning methods, and determines the positive sequential pattern by obtaining the support of the candidate sequences based on the pattern of repeated database scanning. GSP algorithm is a typical algorithm similar to the Apriori. The GSP algorithm, on the basis of the Apriori algorithm, has added classification hierarchy, time constraint and sliding time window technologies to optimize the algorithm as a whole. Also, GSP has also imposed restrictions on the scanning conditions of data sets, which can reduce the number of candidate sequences to be scanned, and reduced the generation of useless patterns.

4. Complex plane, also referred to as complex number plane, is namely z=a+bi whose corresponding coordinate is (a,b), wherein a represents the x-coordinate in the complex plane while b represents the y-coordinate in the complex plane. As all points represent the real number a fall on the x-axis, the x-axis is also referred to as “real axis”; as all points that represent the pure imaginary number b fall on the y-axis, the y-axis is also referred to as “imaginary axis”; there is one and only one real point on the y-axis, namely the origin “0”.

5. Purine Pyrimidine Graph is simply to draw vectors on a plane and show exactly the different base pairs in a DNA sequence. Here, we construct a Purine Pyrimidine Graph on the complex plane with the first and second quadrants showing purines (A, ¬A, G and ¬G) and the third and fourth quadrant showing pyrimidines (T, ¬T, C and ¬C). The unit vectors representing the four nucleotides A, G, C, and T and their corresponding negative sequences are as follows. In this way, different base pairs can be uniquely represented, and the base pairs are conjugate. Such a Purine Pyrimidine Graph can enable the one-to-one correspondence of the DNA sequence to its time sequence.

6. DTW (Dynamic Time Warping) is a nonlinear programming technique that combines time planning and distance measure, and is used to calculate the maximum similarity between two time sequences namely the minimum distance. Its appearance is for a relatively simple purpose, and it has been widely used in the field of speech recognition.

7. Apriori's character indicates that all non-empty subsets of any frequent item set must also be frequent.

### The Technical Solution of the Invention is as Follows

A similarity analysis method of negative sequential patterns based on biological sequences, which comprises steps as follows:

(1) Data preprocessing

Each sequence or genome to be processed must be preprocessed prior to frequent pattern mining. The specific process is as follows: represent the letters in the DNA sequence with numbers; as the DNA sequence is very long, divide the sequence represented by numbers into several blocks each with the same number of bases, and the several blocks obtained shall be used as datasets for frequent pattern mining;

(2) Frequent pattern mining

Utilize the f-NSP algorithm to mine the data sets to obtain the maximum frequent positive and negative sequential patterns;

(3) Represent the maximum frequent positive and negative sequential patterns graphically;

(4) Similarity analysis of DNA sequence

Calculate the similarity of different DNA sequences. The smaller the similarity is, the more similar the DNA sequences are.

A similarity matrix can be used to evaluate the effectiveness of the DNA similarity analysis algorithm, thus shedding light on the evolutionary or genetic relationships between different species. The calculation of the distance between DNA sequences is the basis of DNA similarity analysis, and Euclidean distance and correlation angle are the two most commonly used distance calculation methods. The smaller the Euclidean distance between sequences is, the more similar the DNA sequences are. The smaller the correlation angle between two carriers is, the more similar the DNA sequences are.

According to a preferred embodiment of the invention, the mining of the dataset D with the f-NSP algorithm in Step (2) comprises steps as follows:
A. Obtain all positive frequent sequences with the GSP algorithm and store the bitmap corresponding to each positive frequent sequence in the hash table, including:
   a. Storing all sequence patterns with a length of 1 obtained by scanning the dataset in the original seed set P₁;
   b. Obtain sequence patterns with a length of 1 from the original seed set P₁ and generate a set C2 of candidate sequences with a length of 2 through join operations; prune the candidate sequence set C2 by using the Apriori's character and determine the support of the remaining sequences through scanning the candidate sequence set C₂; store the sequence patterns with support being larger than the minimum support, and output them as sequence pattern L₂ with a length of 2 and take them as a seed set with a length of 2; then, generate candidate sequences of increasing length. Based on this method, output sequence pattern L3 of length 3, sequence pattern L4 of length 4 . . . sequence pattern Ln+1 of length n+1, until no new sequence patterns can be mined. Then, all the positive frequent sequences can be obtained. The minimum support is a user-set value, represented as min_sup. The whole process can be described as follows:

L₁→C₂→L₂→C₃→L₃→C₄→L₄ . . . . Stop if Lₙ₊₁ cannot be generated.

Generate the corresponding NSCs based on all the positive frequent sequences;

NSC refers to a negative candidate sequence, while positive frequent sequences are collectively referred to as positive sequences. To generate all non-redundant NSCs from positive sequences, the key process of generating NSCs is to convert the discontinuous elements with positive patterns into their negative partners. For a k-size PSP, its NSCs are generated by changing any m non-adjacent elements to their negative numbers (represented by ¬), wherein m=1, 2, . . . , ┌k/2┐, ┌k/2┐ is the smallest positive integer not smaller than k/2, and k-size means that the size of the sequence is k. Taking the sequence S={A T T C C} as an example, its size is 5-size. NSCs refer to all negative candidate sequences.

For example, the NSCs of <A T C C> include: (1) <¬AT C C> when m=1, <¬AT C C>, <A ¬T C C>, <AT ¬C C>, <ATC ¬C>; (2) m=2 [symbol] , <¬AT ¬C C>, <A ¬T C ¬C>. The rule here is that two consecutive negative items are not allowed.

C. Calculate the support of the negative candidate sequences quickly by bit operations.

Calculate the support of the NSCs after they are generated. Negative frequent sequence patterns are obtained when the support of negative candidate sequences is satisfied. The support of NSCs shall be calculated as follows: for a given m-size and n-neg-size negative sequence ns, if ∀1-negMSᵢ∈1-negMSₙₛ, 1≤i≤n, then the support of ns in dataset D is:

sup(ns)=sup(MPS(ns))−N(ORᵢ₌₁ⁿ {B(p(1-negMSᵢ))}), where m-size means that the size of the sequence is m. Assuming that ns=<a₁a₂ . . . aₘ> is a negative sequence, if ns' is made up of all the positive elements in ns, then ns' is referred to as the largest positive subsequence of ns, which is denoted as MPS(ns). For example, MPS(<¬T C G ¬A>)=<CG>. The sequence consisting of MPS(ns) and a negative element a in ns is referred to as the maximum 1-neg-size sub-sequence, which is defined as 1-negMS. Taking <¬ATC¬G> as an example, its 1-negMS is <¬ATC> and <TC¬G>.

Through frequent pattern mining, 12 maximum frequent positive and negative sequential patterns are obtained;

According to a preferred embodiment of the invention, the graphical representation of the maximum frequent positive and negative sequential patterns in Step (3) include: constructing a Purine Pyrimidine Graph on the complex plane with first and second quadrants representing the purines, including A, ¬A, G, and ¬G, and the third and fourth quadrants representing pyrimidines, including T, ¬T, C, and ¬C. The four nucleotides A, G, T, and C and their corresponding negative sequence unit vectors ¬A, ¬G, ¬T, and ¬C are as shown in equations (I) to (VIII):

(b+di)→A (I)

(d+bi)→G (II)

(b−di)→T (III)

(d−bi)→C (IV)

(−b−di)→¬A (V)

(−d−bi)→¬G (VI)

(−b+di)→¬T (VII)

(−d+bi)→¬C (VIII)

Where: b and d are non-zero real numbers and

$$b = \frac{1}{2} , d = \frac{\sqrt{3}}{2} ;$$

A and T are conjugate and G and C are also conjugate, namely Ā=T and C̅=G. A, T, C, and G represent the actually existing base pairs while ¬A, ¬T, ¬C, and ¬G represent the base pairs that should be present but are not present in the DNA sequence, also known as missing base pairs or unit vectors of A, G, T, C, and their corresponding negative sequences.

With this representing method, the base \vec{p}ₙ of a DNA sequence can be reduced to a number sequence s(n), as shown in the equation (IX):

$$s ( n ) = s ( 0 ) + {\sum }_{j = 1}^{n} y ( j ) \qquad ( IX )$$

Where: s(0)=0 and y(j) satisfies the equation (X):

$$y ( j ) = \{ \begin{matrix}\frac{1}{2} + \frac{\sqrt{3}}{2} i , if j = A , \\ \frac{\sqrt{3}}{2} + \frac{1}{2} i , if j = G , \\ \frac{1}{2} - \frac{\sqrt{3}}{2} i , if j = T , \\ \frac{\sqrt{3}}{2} - \frac{1}{2} i , if j = C , \\ - \frac{1}{2} - \frac{\sqrt{3}}{2} i , if j = ⫬ A , \\ - \frac{\sqrt{3}}{2} - \frac{1}{2} i , if j = ⫬ G , \\ - \frac{1}{2} + \frac{\sqrt{3}}{2} i , if j = ⫬ T , \\ - \frac{\sqrt{3}}{2} + \frac{1}{2} i , if j = ⫬ C ,\end{matrix} \qquad ( X )$$

Where: j represents the base type in the 0, 1st, 2nd . . . , and nth positions of the sequence; n represents the length of the DNA sequence studied;

The time sequence of the original DNA sequence can be uniquely obtained from the “Purine Pyrimidine Graph” through the above steps.

Convert the 12 kinds of maximum frequent positive and negative sequential patterns into number sequences with the equation (X). Taking the sequence Human1 as an example, the complex number sequence obtained by equations (IX)-(X) is s(H1)={0.866+0.50.366-0.366i, 2.2321+0.134i, 3.0981+0.634i, 3.5981+1.5i, 4.4641+2i} and the time sequence formed is S(H1)={1.0000, 1.4142, 2.2361, 3.1623, 3.8982, 4.8916}. In this way, the time sequences after the transformation of the 12 frequent sequential patterns can be obtained.

According to a preferred embodiment of the invention, a distance matrix used to indicate the similarity of different DNA sequences is calculated and obtained in Step (4)

According to a preferred embodiment of the invention, the distance matrix is calculated by the DTW algorithm in Step (4). Let the time sequences obtained through the transformation of the DNA sequences be S¹(t)={s₁¹, s₂¹, . . . , sₘ¹} and S²(t)={s₁²; s₂², . . . ; sₙ²}; and their length be m and n respectively; sort them according to their time positions and construct a m×n matrix A_{m×n}, with each element in the matrix aᵢⱼ=d(sᵢ¹, sⱼ²)=\sqrt{(sᵢ¹−sⱼ²)²}; in the matrix, the set formed by a group of adjacent matrix elements is referred to as a warping path, which is denoted as W=w₁, w₂, . . . , wₖ, wherein the kth element of W wₖ=(aᵢⱼ)ₖ. Such a path fulfills the following conditions:

max{m,n}≤K≤m+m−1; \textcircled{1}

w₁=a₁₁,wₖ=aₘₙ; \textcircled{2}

For wₖ=aᵢⱼ,wₖ₋ᵢ=aᵢⱼ if 0≤i−i′≤1,0≤j−j′≤1 are satisfied, \textcircled{3}

then

$$DT W ( {S}^{1} , {S}^{2} ) = min ( \frac{1}{k} \sqrt{{\sum }_{i = 1}^{k} {w}_{i}} ) .$$

The DTW algorithm applies the idea of dynamic programming to find the best path with the least warping cost, as shown in equation (XI):

$$\{ \begin{matrix}D ( 1 , 1 ) = {a}_{11} \\ D ( i , j ) = {a}_{ij} + min \{ D ( i - 1 , j - 1 ) , D ( i , j - 1 ) , D ( i - 1 , j ) \}\end{matrix} \qquad ( XI )$$

Where: i=2, 3, . . . , m; j=2, 3, . . . , n. D(m,n) is the minimum cumulative value of the warping path in A_{m×n}. . . .

The above implementation system of the similarity analysis method comprises data preprocessing module, frequent pattern mining module, graphical representation module, and similarity analysis module which are sequentially connected. The said data preprocessing module is used to execute Step (1); the said frequent pattern mining module is used to execute Step (2); the said graphical representation module is used to execute Step (3); and the similarity analysis module is used to execute Step (4).

A computer-readable storage medium, which is characterized in that it stores the similarity analysis programs of negative sequential patterns based on biological sequences. The said similarity analysis programs can realize the steps of any one of the said similarity analysis methods of the negative sequential patterns based on biological sequences.

### The Beneficial Effects of the Invention are as Follows

1. The invention can express and analyze the negative sequences effectively, and obtain different analysis results by selecting different combinations of maximum frequent patterns.
2. The invention selects frequent patterns for similarity analysis, which can save computer memory and time consumption greatly.

### BRIEF DESCRIPTION OF THE FIGURES

FIG. 1 is the flow block diagram of the similarity analysis method of negative sequential patterns based on biological sequences in the invention;

FIG. 2 is the diagram of the Purine Pyrimidine Graph in the invention;

FIG. 3 is the structure block diagram of the implementation system for the similarity analysis method of negative sequential patterns based on biological sequences in the invention;

FIG. 4 is the schematic diagram of the bitwise OR operation process in the embodiments;

FIG. 5(a) is the phylogenetic tree diagram drawn after conducting similarity analysis on the maximum frequent sequences Human1, Opossum2, Rat2 and Chimpanzee2;

FIG. 5(b) is the phylogenetic tree diagram drawn after conducting similarity analysis on the maximum frequent sequences Human2, Opossum1, Rat2, and Chimpanzee1;

FIG. 6(a) is the phylogenetic tree diagram drawn after conducting similarity analysis on the maximum frequent sequences Human2, Opossum2, Rat2 and Chimpanzee1;

FIG. 6(b) is the phylogenetic tree diagram drawn after conducting similarity analysis on the maximum frequent sequences Human3, Opossu3, Rat3 and Chimpanzee3;

FIG. 7 is the distance diagram of the normalized species.

### DETAILED EMBODIMENTS

The invention is further described in combination with the attached figures and embodiments as follows, but is not limited to that.

### Embodiment 1

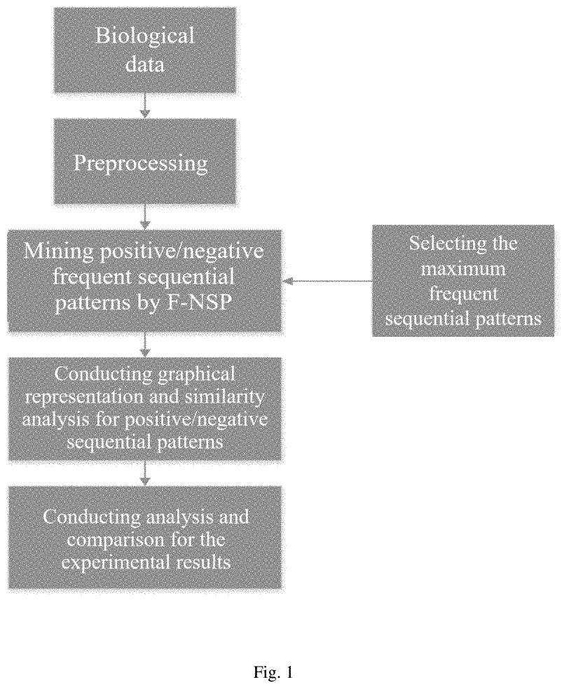

A similarity analysis method of negative sequential patterns based on biological sequences, as shown in FIG. 1, which comprises steps as follows:

(1) Data preprocessing

Each sequence or genome to be processed must be preprocessed prior to frequent pattern mining. The specific process is as follows: represent the letters in the DNA sequence with numbers; as the DNA sequence is very long, divide the sequence represented by numbers into several blocks each with the same number of bases, and the several blocks obtained shall be used as datasets for frequent pattern mining;

In the present invention, each sequence is first divided into several blocks, with each block consisting of the same number of continuous bases. The blocks are independent of each other, and the size of the blocks can be changed in practice. However, one thing needing to be noted is that if the size of the last block is smaller than that of the specified block, the block will be discarded. For clarity, here's an example of a segmentation block. There are two sequences, respectively S₁ and S₂ in the example. Assuming that the block size is 15 and the two sequences are divided into two and three blocks, respectively, then the last block of size 3 will be discarded. Each of these blocks is marked with a curve and line. Such a process is also known as sequence blocking. It is an important step, and it brings two main benefits. First, it can capture fine-grained information about a sequence, including positional information and sequencing information. Second, it can reduce memory and time consumption for sequence processing, even for long sequences.

| | |
|---|---|
| S1 | [symbol] ACCTGGACCCTTGAT (SEQ ID NO: 01) |
| S2 | [symbol] ACCTGGACCCTTGAT [symbol] [symbol] |
| | (SEQ ID NO: 02) |

Currently, few DNA sequences can be used for sequence similarity studies, and it remains an issue to find more suitable DNA sequences. The three exon sequences of the hemoglobin genes from 15 species are the most commonly used DNA sequences. The three gene sequences, consisting of the first, second and third exons, have an average length of 92 bases, 222 bases and 114 bases, respectively. Among them, the first exons of the β genes from 11 different species are the most widely used DNA sequence data.

The selected data set comprises the first exons of the β protein genes from four species, as shown in Table 1:

TABLE 1

| | |
|---|---|
| Human | ATGGTGCACCTGACTCCTGAGGAGAAGTCTGCCGTTA |
| | CTGCCCTGTGGGGCAAGGTGAACGTGGATTAAGTTGG |
| | TGGTGAGGCCCTGGGCAG (SEQ ID NO: 03) |
| Opossum | ATGGTGCACTTGACTTCTGAGGAGAAGAACTGCATCA |
| | CTACCATCTGGTCTAAGGTGCAGGTTGACCAGACTGG |
| | TGGTGAGGCCCTTGGCAG (SEQ ID NO: 04) |
| Rat | ATGGTGCACCTAACTGATGCTGAGAAGGCTACTGTTA |
| | GTGGCCTGTGGGGAAAGGTGAACCCTGATAATGTTGG |
| | CGCTGAGGCCCTGGGCAG (SEQ ID NO: 05) |
| Chimpanzee | ATGGTGCACCTGACTCCTGAGGAGAAGTCTGCCGTTA |
| | CTGCCCTGTGGGGCAAGGTGAACGTGGATGAAGTTGG |
| | TGGTGAGGCCCTGGGCAGGTTGGTATCAAGG |
| | (SEQ ID NO: 06) |

(2) Frequent pattern mining

Utilize the f-NSP algorithm to mine the data sets to obtain the maximum frequent positive and negative sequential patterns;

(3) Represent the maximum frequent positive and negative sequential patterns graphically;

(4) Similarity analysis of DNA sequence

Calculate the similarity of different DNA sequences. The smaller the similarity is, the more similar the DNA sequences are.

A similarity matrix can be used to evaluate the effectiveness of the DNA similarity analysis algorithm, thus shedding light on the evolutionary or genetic relationships between different species. The calculation of the distance between DNA sequences is the basis of DNA similarity analysis, and Euclidean distance and correlation angle are the two most commonly used distance calculation methods. The smaller the Euclidean distance between sequences is, the more similar the DNA sequences are. The smaller the correlation angle between two carriers is, the more similar the DNA sequences are.

### Embodiment 2

A similarity analysis method of negative sequential patterns based on biological sequences according to Embodiment 1, provided however that:

The mining of the dataset D with the f-NSP algorithm in Step (2) comprises steps as follows:
A. Obtain all positive frequent sequences with the GSP algorithm and store the bitmap corresponding to each positive frequent sequence in the hash table, including:
   a. Storing all sequence patterns with a length of 1 obtained by scanning the dataset in the original seed set P₁;
   b. Obtain sequence patterns with a length of 1 from the original seed set P₁ and generate a set C2 of candidate sequences with a length of 2 through join operations; prune the candidate sequence set C2 by using the Apriori's character and determine the support of the remaining sequences through scanning the candidate sequence set C2; store the sequence patterns with support being larger than the minimum support, and output them as sequence pattern L₂ with a length of 2 and take them as a seed set with a length of 2; then, generate candidate sequences of increasing length. Based on this method, output sequence pattern L3 of length 3, sequence pattern L4 of length 4 . . . , and sequence pattern Ln+1 of length n+1, until no new sequence patterns can be mined. Then, all the positive frequent sequences can be obtained. The minimum support is a user-set value, represented as min_sup. The whole process can be described as follows:

L₁→C₂→L₂→C₃→L₃→C₄→L₄ . . . . Stop if Lₙ₊₁ cannot be generated.

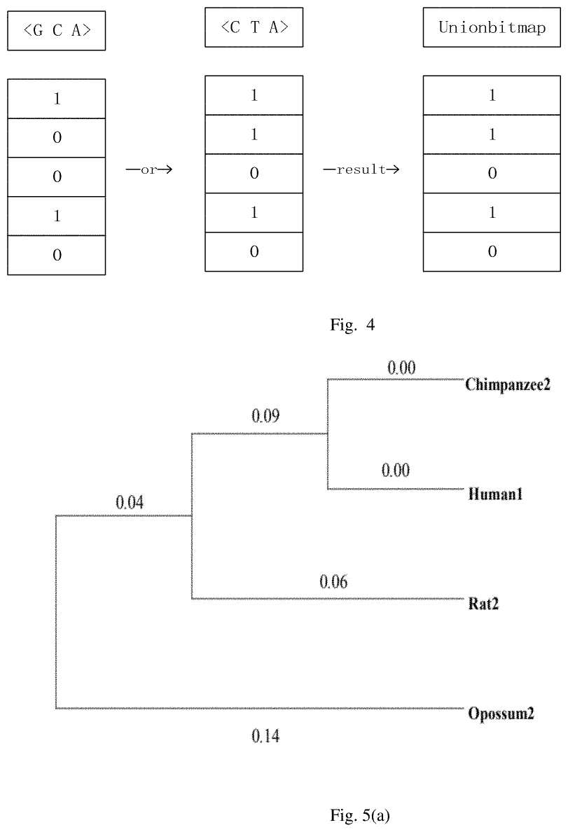

FIG. 4 is used to explain the bitwise OR operations. For sequence S, if sup(s)≥min_sup, it is referred to as a frequent (positive) sequential pattern, while if sup(s)<min_sup, it is an infrequent sequential pattern. Let a positive frequent sequence be <G C T A> and sup (C A)=5, and then ns, one of the negative candidate sequences, can be <¬GC ¬TA> according to the negative candidate sequence generation method. Accordingly, MPS(ns)=<CA>, P(1-negMS₁)=<GCA>, and P(1-negMS₂)=<C TA>. Let B (<G CA>)=|1|0|0|1|0| and B (<C TA>)=|1|1|0|1|0|, and then the bitmap of B(<GCA>)ORB(<CTA>) is as shown in FIG. 4. Thus, it can be easily known that N(unionbitmap)=4 and, according to formula 1, sup (<¬GC ¬TA>)=1.

Generate the corresponding NSCs based on all the positive frequent sequences;

NSC refers to a negative candidate sequence, while positive frequent sequences are collectively referred to as positive sequences. To generate all non-redundant NSCs from positive sequences, the key process of generating NSCs is to convert the discontinuous elements with positive patterns into their negative partners. For a k-size PSP, its NSCs are generated by changing any m non-adjacent elements to their negative numbers (represented by ¬), wherein m=1, 2, . . . , ┌k/2┐, ┌k/2┐ is the smallest positive integer not smaller than k/2, and k-size means that the size of the sequence is k. Taking the sequence S={A T T C C} as an example, its size is 5-size. NSCs refer to all negative candidate sequences.

For example, the NSCs of <A T C C> include: (1)<¬AT C C>, <A ¬T C C>, <AT ¬C C>, and <ATC ¬C> when m=1; (2) <¬AT ¬C C>, <A ¬T C ¬C> when m=2. The rule here is that two consecutive negative items are not allowed.

C. Calculate the support of the negative candidate sequences quickly by bit operations.

Calculate the support of the NSCs after they are generated. Negative frequent sequence patterns are obtained when the support of negative candidate sequences is satisfied. The support of NSCs shall be calculated as follows: for a given m-size and n-neg-size negative sequence ns, if ∀1-negMSᵢ∈1-negMSₙₛ, 1≤i≤n, then the support of ns in dataset D is:

sup(ns)=sup(MPS(ns))−N(ORᵢ₌₁ⁿ{B(p(1-negMSᵢ))}), where m-size means that the size of the sequence is m. Assuming that ns=<a₁a₂ . . . aₘ> is a negative sequence, if ns' is made up of all the positive elements in ns only, then ns' is referred to as the largest positive subsequence of ns, which is denoted as MPS(ns). For example, MPS(<¬T C G ¬A>)=<CG>. The sequence consisting of MPS(ns) and a negative element a in ns is referred to as the maximum 1-neg-size sub-sequence, which is defined as 1-negMS. Taking <¬ATC¬G> as an example, its 1-negMS is <¬ATC> and <TC¬G>.

Through frequent pattern mining, 12 maximum frequent positive and negative sequential patterns are obtained;

Maximal frequent sequential pattern. Given a DNA sequence, also a base sequence, S=<s₁ s₂ . . . sₙ>, where sᵢ(1≤i≤n) is a character set of the character Ω={A [symbol] T [symbol] C [symbol] G}, if the support of a pattern <sₖ sₖ₊₁ . . . sₘ> (1≤k≤m≤n) is no smaller than the minimum support, then the sequence is a frequent sequence. A maximum frequent pattern is a pattern whose super sequences are infrequent. Let min_sup=0.3 and obtain multiple maximum frequent sequential patterns. 12 frequent sequential patterns are selected from among them as data sets for sequential pattern analysis. The 12 frequent sequential patterns are as shown in Table 2 below:

TABLE 2

| | |
|---|---|
| Human1 | GTGGAG |
| Human2 | GGGGGA |
| Human3 | ¬AGTG¬CGA¬CG |
| Opossum1 | GGCGCA |
| Opossum2 | GGCTTA |
| Opossum3 | GGC¬GGCA¬G |
| Rat1 | GCCTGA |
| Rat2 | GGTGGG |
| Rat3 | GCC¬ATGA¬C |
| Chimpanzee1 | GGGGAG |
| Chimpanzee2 | GTGGAG |
| Chimpanzee3 | ¬AGGG¬CGAG |

### 1. Embodiment 3

A similarity analysis method of negative sequential patterns based on biological sequences according to Embodiment 1, provided however that:

The graphical representation of the maximum frequent positive and negative sequential patterns in Step (3) include: constructing a Purine Pyrimidine Graph on the complex plane with first and second quadrants representing the purines, including A, ¬A, G, and ¬G, and the third and fourth quadrants representing pyrimidines, including T, ¬T, C, and ¬C. The four nucleotides A, G, T, and C and their corresponding negative sequence unit vectors ¬A, ¬G, ¬T, and ¬C are as shown in equations (I) to (VIII):

(b+di)→A (I)

(d+bi)→G (II)

(b−di)→T (III)

(d−bi)→C (IV)

(−b−di)→¬A (V)

(−d−bi)→¬G (VI)

(−b+di)→¬T (VII)

(−d+bi)→¬C (VIII)

Where: b and d are non-zero real numbers and

$$b = \frac{1}{2} and d = \frac{\sqrt{3}}{2} ;$$

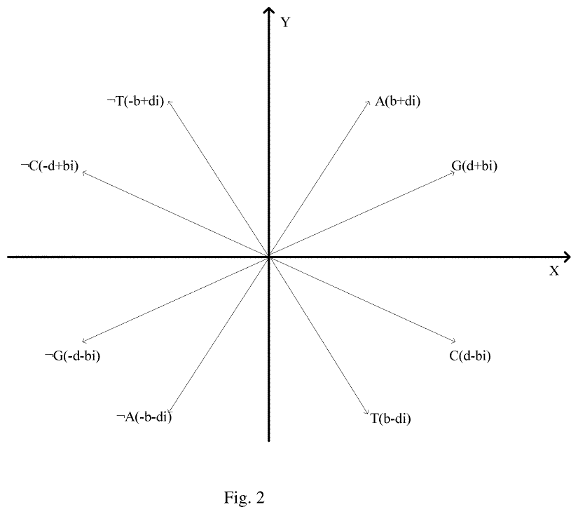

A and T are conjugate and G and C are also conjugate, namely Ā=T and C̅=G. A, T, C, and G represent the actually existing base pairs while ¬A, ¬T, ¬C, and ¬G represent the base pairs that should be present but are not present in the DNA sequence, also known as missing base pairs or unit vectors of A, G, T, C and their corresponding negative sequences, as shown in FIG. 2.

With this representing method, the base \vec{p}ₙ of a DNA sequence can be reduced to a number sequence s(n), as shown in the equation (IX):

$$s ( n ) = s ( 0 ) + {\sum }_{j = 1}^{n} y ( j ) \qquad ( I X )$$

Where: s(0)=0 and y(j) satisfies the equation (X):

$$y ( j ) = \{ \begin{matrix}\frac{1}{2} + \frac{\sqrt{3}}{2} i , if j = A , \\ \frac{\sqrt{3}}{2} + \frac{1}{2} i , if j = G , \\ \frac{1}{2} - \frac{\sqrt{3}}{2} i , if j = T , \\ \frac{\sqrt{3}}{2} - \frac{1}{2} i , if j = C , \\ - \frac{1}{2} - \frac{\sqrt{3}}{2} i , if j = ⫬ A , \\ - \frac{\sqrt{3}}{2} - \frac{1}{2} i , if j = ⫬ G , \\ - \frac{1}{2} + \frac{\sqrt{3}}{2} i , if j = ⫬ T , \\ - \frac{\sqrt{3}}{2} + \frac{1}{2} i , if j = ⫬ C ,\end{matrix} \qquad ( X )$$

Where: j represents the base type in the 0, 1st, 2nd . . . , and nth positions of the sequence; n represents the length of the DNA sequence studied;

The time sequence of the original DNA sequence can be uniquely obtained from the “Purine Pyrimidine Graph” through the above steps.

Convert the 12 kinds of maximum frequent positive and negative sequential patterns into number sequences with the equation (X). Taking the sequence Human1 as an example, the complex number sequence obtained by equations (IX)-(X) is s(H1)={0.866+0.5i, 1.366-0.366i, 2.2321+0.134i, 3.0981+0.634i, 3.5981+1.5i, 4.4641+2i}, and the time sequence formed is S(H1)={1.0000, 1.4142, 2.2361, 3.1623, 3.8982, 4.8916}. In this way, the time sequences after the transformation of the 12 frequent sequential patterns can be obtained.

### 1. Embodiment 4

A similarity analysis method of negative sequential patterns based on biological sequences according to Embodiment 1, provided however that:

A distance matrix used to indicate the similarity of different DNA sequences is calculated and obtained in Step (4) with the DTW algorithm.

Let the time sequences obtained through the transformation of the DNA sequences be S¹(t)={s₁¹, s₂¹, . . . , sₘ¹} and S²(t)={s₁², s₂², . . . , sₙ²}, and their length be in and n respectively; sort them according to their time positions and construct a m×n matrix A_{m×n}, with each element in the matrix aᵢⱼ=d(sᵢ¹, sⱼ²)=\sqrt{(sᵢ¹−sⱼ²)²}; in the matrix, the set formed by a group of adjacent matrix elements is referred to as a warping path, which is denoted as W=w₁,w₂, . . . , wₖ, wherein the kth element of W wₖ=(aᵢⱼ)ₖ. Such a path fulfills the following conditions:

max{m,n}≤K≤m+m−1; \textcircled{1}

w₁=a₁₁,wₖ=aₘₙ; \textcircled{2}

For wₖ=aᵢⱼ,wₖ₋₁=aᵢⱼ if 0≤i−i′≤1,0≤j−j′≤1 are satisfied, \textcircled{3}

then

$$DT W ( {S}^{1} , {S}^{2} ) = min ( \frac{1}{k} \sqrt{{\sum }_{i = 1}^{k} {w}_{i}} ) .$$

The DTW algorithm applies the idea of dynamic programming to find the best path with the least warping cost, as shown in equation (XI):

$$\{ \begin{matrix}D ( 1 , 1 ) = {a}_{11} \\ D ( i , j ) = {a}_{ij} + min \{ D ( i - 1 , j - 1 ) , D ( i , j - 1 ) , D ( i - 1 , j ) \}\end{matrix} \qquad ( XI )$$

Where: i=2, 3, . . . , m; j=2, 3, . . . , n. D(m,n) is the minimum cumulative value of the warping path in A_{m×n}. . . .

DTW distance measurement is performed on the time sequences transformed from the 12 frequent sequences and the distance matrixes between the 8 PSPs and the 4 NSPs are obtained respectively, as shown in Table 3 and Table 4:

TABLE 4

| SP | Human3 | Opossum3 | Rat3 | Chimpanzee3 |
|---|---|---|---|---|
| Human3 | 0 | 0.4116 | 0.4352 | 0.2068 |
| Opossum3 | | 0 | 0.1547 | 0.5324 |
| Rat3 | | | 0 | 0.6632 |
| Chimpanzee3 | | | | 0 |

It is understood that Humans and Chimpanzees are primates, rats are rodents, and opossums are metatherian animals. The overall variations shown by the method in the present invention are consistent with the classification, so the method proposed in the invention is effective and feasible. Moreover, the proposed method is effective for both short and long sequences. Since the data used in the present invention is the frequent patterns after mining, and the length of the sequences used for comparison is generally shortened, but the characteristics of the original sequences are retained, the calculation is very simple and the computer memory consumption is saved. By comparing the similarities between the four species, it can be known that the combination of different patterns can produce different results, which may be useful under different considerations.

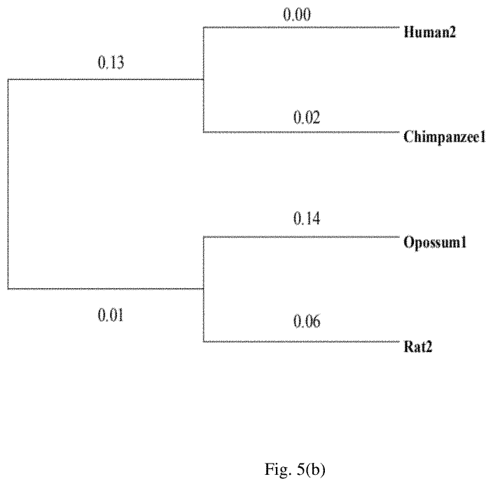

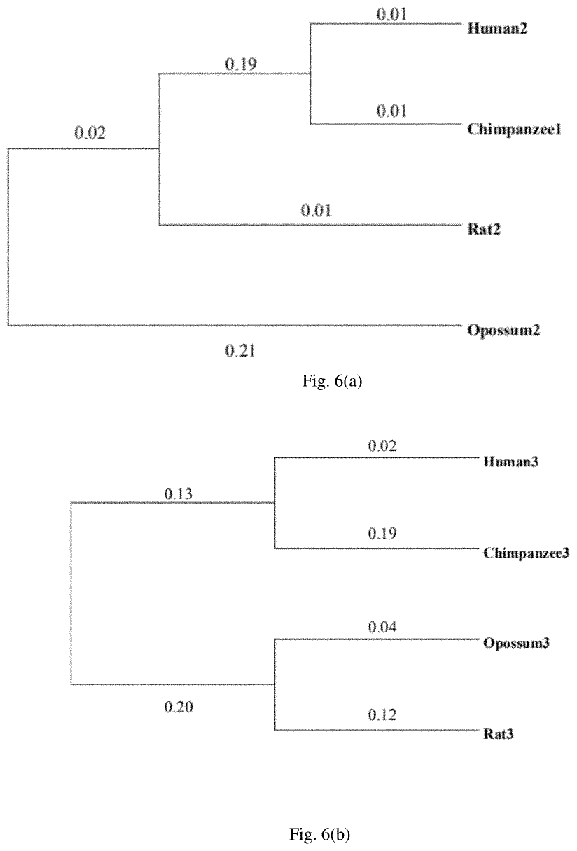

A number of maximum frequent sequences and their distance matrixes (as shown in Table 3 and Table 4) are randomly selected. The similarity of different data groups is listed in Table 3 and Table 4. If clustering can be carried out reasonably, the phylogenetic tree can be constructed by using the method in the invention. The Molecular Evolutionary Genetics Analysis Version 5.0 (MEGA5) is a user-friendly software for building sequence alignment and phylogenetic trees. A phylogenetic tree is a tree-shaped branching diagram that summarizes the genetic or evolutionary relationships of various creatures. FIG. 5(a) is the phylogenetic tree diagram drawn after conducting similarity analysis on the maximum frequent sequences Human1, Opossum2, Rat2 and Chimpanzee2; FIG. 5(b) is the phylogenetic tree diagram drawn after conducting similarity analysis on the maximum frequent sequences Human2, Opossum1, Rat2, and Chimpanzee1; FIG. 6(a) is the phylogenetic tree diagram drawn after conducting similarity analysis on the maximum frequent sequences Human2, Opossum2, Rat2 and Chimpanzee1; FIG. 6(b) is the phylogenetic tree diagram drawn after conducting similarity analysis on the maximum frequent sequences Human3, Opossu3, Rat3 and Chimpanzee3. The invention obtains four different classification results by selecting four combinations of frequent patterns, which all conform to the evolutionary laws of species.

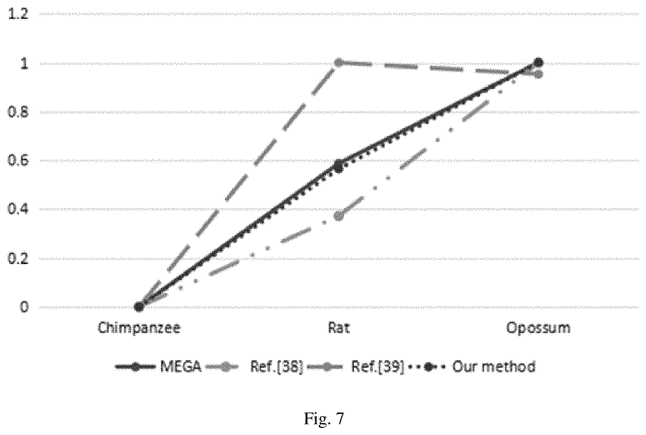

By normalizing the data, the results of the invention are compared with those of the other methods. FIG. 7 is the normalized distance diagram of the species, wherein the y-ordinate represents the normalized distance. FIG. 7 shows the Pearson correlation coefficients between the results of this method and two comparative methods and the MEGA results. Table 5 details the distance from other species and humans of the four methods.

TABLE 5

| | | | | Correlation |
|---|---|---|---|---|
| | Chimpanzee | Rat | Opossum | coefficient |
| MEGA | 0.0095 | 0.4935 | 0.8337 | |
| | (0.0000) | (0.5872) | (1) | |
| Ref.Error! Reference | 0.0309 | 0.1198 | 0.2696 | 0.9697 |
| source not found. | (0) | (0.3724) | (1) | |
| Ref.Error! Reference | 5.3704 | 27.0102 | 25.9952 | 0.8939 |
| source not found. | (0) | (1) | (0.9531) | |
| Our method | 0.0000 | 0.1547 | 0.2739 | 0.9997 |
| | | (0.5648) | (1) | |

In Table 5, the values in parentheses are the true distance after normalization to 0 to 1. The Pearson correlation coefficient between this method and the two comparative methods is calculated by reference to ZhiyiMo, WenZhu, Yi Sun, Qilin Xiang, MingZheng, MinChen, ZejunLi. One novel representation of DNA sequence based on the global and local position information.[J]. Scientific reports,2018,8(1). Ref.Error! Reference source not found. Yu Hong-Jie, Huang De-Shuang. Graphical representation for DNA sequences via joint diagonalization of matrix pencil.[J]. IEEE Journal of Biomedical & Health Informatics, 2013, 17(3):503-511.As can be seen from the table, the method in the invention has the highest correlation coefficient with MEGA, indicating that the method can more accurately calculate the similarity between DNA sequences. In addition, it can be seen from FIG. 7 that the method is closer to the curve calculated by MEGA, which again indicates that the method has the highest correlation with MEGA.

The comparison shows that the method in the invention can express and analyze the negative sequences effectively and can obtain different analysis results by selecting different combinations of maximum frequent patterns. As frequent patterns are selected for similarity analysis, the computer memory and time consumption can be greatly saved. This method also has the highest correlation with MEGA.

### Embodiment 5

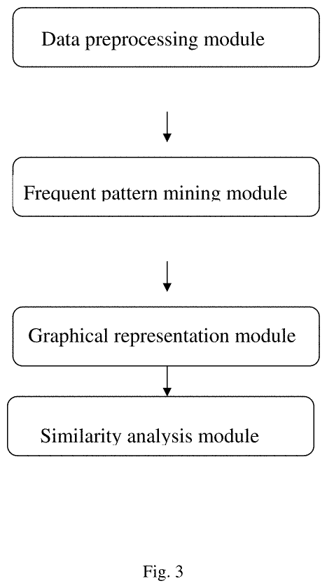

An implementation system for the similarity analysis method of negative sequential patterns based on biological sequences according to any one of Embodiments 1-4, which, as shown in FIG. 3, comprises data preprocessing module, frequent pattern mining module, graphical representation module, and similarity analysis module which are sequentially connected. The said data preprocessing module is used to execute Step (1); the said frequent pattern mining module is used to execute Step (2); the said graphical representation module is used to execute Step (3); and the similarity analysis module is used to execute Step (4).

### Embodiment 6

A computer-readable storage medium, which is characterized in that it stores the similarity analysis programs of negative sequential patterns based on biological sequences. The said similarity analysis programs of negative sequential patterns based on biological sequences can realize the steps of the similarity analysis method of negative sequential patterns based on biological sequences in any one of Embodiments 1-4.

What is claimed is:

1. A similarity analysis method of negative sequential patterns based on biological sequences, which is characterized in that it comprises steps as follows:
(1) data preprocessing
represent the letters in the DNA sequence with numbers; as the DNA sequence is very long, divide the sequence represented by numbers into several blocks each with the same number of bases, and the several blocks obtained shall be used as datasets for frequent pattern mining;
(2) frequent pattern mining
utilize the f-NSP algorithm to mine the data sets to obtain the maximum frequent positive and negative sequential patterns;
(3) represent the maximum frequent positive and negative sequential patterns graphically;
(4) similarity analysis of DNA sequence
calculate the similarity of different DNA sequences. The smaller the similarity is, the more similar the DNA sequences are.

2. The similarity analysis method of negative sequential patterns based on biological sequences according to claim 1, which is characterized in that the mining of the dataset D with the f-NSP algorithm in Step (2) comprises steps as follows:
A) obtain all positive frequent sequences with the GSP algorithm and store the bitmap corresponding to each positive frequent sequence in the hash table, including:
a) storing all sequence patterns with a length of 1 obtained by scanning the dataset in the original seed set P₁;
b) obtain sequence patterns with a length of 1 from the original seed set P₁ and generate set C2 of candidate sequences with a length of 2 through join operations; prune the candidate sequence set C2 by using the Apriori's character and determine the support of the remaining sequences through scanning the candidate sequence set C₂; store the sequence patterns with support being larger than the minimum support, and output them as sequence pattern L₂ with a length of 2 and take them as a seed set with a length of 2; based on this method, output sequence pattern L3 of length 3, sequence pattern L4 of length . . . sequence pattern Ln+1 of length n+1, until no new sequence patterns can be mined; then, all the positive frequent sequences can be obtained; the minimum support is a user-set value, represented as min_sup;
B) generate the corresponding NSCs based on all the positive frequent sequences;
NSC refers to a negative candidate sequence, while positive frequent sequences are collectively referred to as positive sequences; for a k-size PSP, its NSCs are generated by changing any m non-adjacent elements to their negative numbers (represented by ¬), wherein m=1, 2, . . . , ┌k/2┐, ┌k/2┐ is the smallest positive integer not smaller than k/2, and k-size means that the size of the sequence is k; NSCs refer to all negative candidate sequences;
C) calculate the support of the negative candidate sequences quickly by bit operations;
the support of NSCs shall be calculated as follows: for a given m-size and n-neg-size negative sequence ns, if ∀1negMSᵢ∈1-negMSₙₛ, 1≤i≤n, then the support of ns in dataset D is:
sup(ns)=sup(MPS(ns))−N(ORᵢ₌₁ⁿ{B(p(1-negMSᵢ))}), where m-size means that the size of the sequence is m; assuming that ns=<a₁a₂ . . . aₘ> is a negative sequence, if ns′ is made up of all the positive elements in ns, then ns′ is referred to as the largest positive subsequence of ns, which is denoted as MPS(ns); the sequence consisting of MPS (ns) and a negative element a in ns is referred to as the maximum 1-neg-size sub-sequence, which is defined as 1-negMS;
through frequent pattern mining, 12 maximum frequent positive and negative sequential patterns are obtained.

3. The similarity analysis method of negative sequential patterns based on biological sequences according to claim 1, which is characterized in that the graphical representation of the maximum frequent positive and negative sequential patterns in Step (3) include: constructing a Purine Pyrimidine Graph in the complex plane with the first and second quadrants representing the purines, including A, ¬A, G, and ¬G, and the third and fourth quadrants representing pyrimidines, including T, ¬T, C, and ¬C. The four nucleotides A, G, T, and C and their corresponding negative sequence unit vectors ¬A, ¬G, ¬T, and ¬C are as shown in equations (I) to (VIII):
(b+di)→A (I)
(d+bi)→G (II)
(b−di)→T (III)
(d−bi)→C (IV)
(−b−di)→¬A (V)
(−d−bi)→¬G (VI)
(−b+di)→¬T (VII)
(−d+bi)→¬C (VIII)
where: b and d are non-zero real numbers and
$$b = \frac{1}{2} , d = \frac{\sqrt{3}}{2} ;$$
A and T are conjugate and G and C are also conjugate, namely Ā=T and C̅=G. A, T, C, and G represent the actually existing base pairs while ¬A, ¬T, ¬C, and ¬G represent the base pairs that should be present but are not present in the DNA sequence, also known as missing base pairs or unit vectors of A, G, T, C and their corresponding negative sequences;
with this representing method, the base \vec{p}ₙ of a DNA sequence can be reduced to a number sequence s(n), as shown in the equation (IX):
$$s ( n ) = s ( 0 ) + {\sum }_{j = 1}^{n} y ( j ) \qquad ( I X )$$
Where: s(0)=0 and y(j) satisfies the equation (X):
$$\begin{matrix}y ( j ) = \{ \begin{matrix}\frac{1}{2} + \frac{\sqrt{3}}{2} i , if j = A , \\ \frac{\sqrt{3}}{2} + \frac{1}{2} i , if j = G , \\ \frac{1}{2} - \frac{\sqrt{3}}{2} i , if j = T , \\ \frac{\sqrt{3}}{2} - \frac{1}{2} i , if j = C , \\ - \frac{1}{2} - \frac{\sqrt{3}}{2} i , if j = ⫬ A , \\ - \frac{\sqrt{3}}{2} - \frac{1}{2} i , if j = ⫬ G , \\ - \frac{1}{2} + \frac{\sqrt{3}}{2} i , if j = ⫬ T , \\ - \frac{\sqrt{3}}{2} + \frac{1}{2} i , if j = ⫬ C ,\end{matrix} & \end{matrix} \qquad ( X )$$
where: j represents the base type in the 0, 1st, 2nd . . . , and nth positions of the sequence; n represents the length of the DNA sequence studied;
convert the 12 maximum frequent positive and negative sequential patterns into number sequences with the equation (X).

4. The similarity analysis method of negative sequential patterns based on biological sequences according to claim 1, which is characterized in that a distance matrix used to indicate the similarity of different DNA sequences is calculated and obtained in Step (4).

5. The similarity analysis method of negative sequential patterns based on biological sequences according to claim 4, which is characterized in that the distance matrix is calculated by the DTW algorithm in Step (4); let the time sequences obtained through the transformation of the DNA sequences be S¹(t)={s₁¹, s₂¹, . . . , sₘ¹} and S²(t)={s₁², s₂², . . . , sₙ²}, and their length be m and n respectively; sort them according to their time positions and construct a m×n matrix A_{m×n}, with each element in the matrix aᵢⱼ=d(sᵢ¹, sⱼ²)=\sqrt{(sᵢ¹−sⱼ²)²}; in the matrix, the set formed by a group of adjacent matrix elements is referred to as a warping path, which is denoted as W=w₁, w₂, . . . , wₖ, wherein the kth element of W wₖ=(aᵢⱼ)ₖ. Such a path fulfills the following conditions:
max{m,n}≤K≤m+m−1; \textcircled{1}
w₁=a₁₁,wₖ=aₘₙ; \textcircled{2}
For wₖ=aᵢⱼ,wₖ₋ᵢ=aᵢⱼ if 0≤i−i′≤1,0≤j−j′≤1 are satisfied, \textcircled{3}
$$DT W ( {S}^{1} , {S}^{2} ) = min ( \frac{1}{k} \sqrt{{\sum }_{i = 1}^{k} {w}_{i}} ) .$$
the DTW algorithm applies the idea of dynamic programming to find the best path with the least warping cost, as shown in equation (XI):
$$\{ \begin{matrix}D ( 1 , 1 ) = {a}_{11} \\ D ( i , j ) = {a}_{ij} + min \{ D ( i - 1 , j - 1 ) , D ( i , j - 1 ) , D ( i - 1 , j ) \}\end{matrix} \qquad ( XI )$$
Where: i=2, 3, . . . , m; j=2, 3, . . . , n; D(m,n) is the minimum cumulative value of the warping path in A_{m×n}.

6. An implementation system for the similarity analysis method of negative sequential patterns based on biological sequences according to claim 1, which is characterized in that it comprises data preprocessing module, frequent pattern mining module, graphical representation module, and similarity analysis module which are sequentially connected. The said data preprocessing module is used to execute Step (1); the said frequent pattern mining module is used to execute Step (2); the said graphical representation module is used to execute Step (3); and the similarity analysis module is used to execute Step (4).

7. A computer-readable storage medium, which is characterized in that it stores the similarity analysis programs of negative sequential patterns based on biological sequences. The said similarity analysis programs of negative sequential patterns based on biological sequences can realize the steps of any one of the similarity analysis methods of negative sequential patterns based on biological sequences according to claim 1.
